(19) United States
(12) Patent Application Publication
(10) Pub. No.: US 2024/0219544 A1
(43) Pub. Date: Jul. 4, 2024

(54) METHOD AND SYSTEM FOR SUPPRESSING GRATING LOBES USING VARIABLE FREQUENCY AS A FUNCTION OF BEAM STEERING

(71) Applicant: GE Precision Healthcare LLC, Wauwatosa, WI (US)

(72) Inventors: Geir Ultveit Haugen, Bærum (NO); Magnar Kopangen Bugge, Horten (NO)

(21) Appl. No.: 18/091,579

(22) Filed: Dec. 30, 2022

(51) Int. Cl. G01S 7/52; A61B 8/00; G01S 15/89

(52) U.S. Cl. CPC G01S 7/52047; A61B 8/56; G01S 7/52092; G01S 15/8952; G01S 15/8915

(57) ABSTRACT

Systems and methods for suppressing grating lobes using variable frequency as a function of beam steering are provided. The method includes transmitting one or more beams each at a beam steering angle. The method includes converting received echoes to generate ultrasound signals corresponding to the one or more beams. The method includes processing the ultrasound signals to generate an ultrasound image. The method includes causing a display system to present the ultrasound image. A transmit frequency and/or a receive frequency is selected based on the beam steering angle of each of the one or more beams. A larger beam steering angle corresponds with a lower transmit frequency and/or a lower receive frequency. A smaller beam steering angle corresponds with a higher transmit frequency and/or a higher receive frequency.

Certain embodiments relate to ultrasound imaging. More specifically, certain embodiments relate to a method and system for suppressing grating lobes using variable frequency as a function of beam steering.

### BACKGROUND

Ultrasound imaging is a medical imaging technique for imaging organs and soft tissues in a human body. Ultrasound imaging uses real time, non-invasive high frequency sound waves to produce a series of two-dimensional (2D), three-dimensional (3D), and/or four-dimensional (4D) images.

Grating lobes are an undesirable portion of an ultrasound beam emitted off axis that produce image artifacts due to error in positioning the returning echo. Grating lobes are caused by a fundamental physical effect in the sense that there are directions, other than the intended steering direction of a given beam, which contribute to the beam in question due to inadequate suppression of incoming energy. Ultrasound images have a large dynamic range, and the presence of grating lobes is particularly noticeable in darker regions of the image where it manifests as shadows or clouds. The level of grating lobes in an ultrasound image depends on probe element spacing and imaging frequency. To avoid grating lobes, the imaging frequency should be kept low enough so that the element spacing is at most half of the wavelength. For frequencies higher than this limit, grating lobes will appear above a maximum beam steering angle. However, higher imaging frequencies provide higher image resolution. Accordingly, a problem exists related to a desire to maximize image resolution while avoiding grating lobes in acquired ultrasound images.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for suppressing grating lobes using variable frequency as a function of beam steering, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to suppress grating lobes using variable frequency as a function of beam steering, in accordance with various embodiments.

FIG. 2 is an exemplary display of an ultrasound image having a grating lobe, in accordance with various embodiments.

FIG. 3 is an exemplary display of an ultrasound image with a suppressed grating lobe as compared to FIG. 2, in accordance with various embodiments.

FIG. 4 is an illustration of an exemplary ultrasound beam transmission at a beam steering angle and received incoming energy providing the grating lobe of FIG. 2, in accordance with various embodiments.

FIG. 5 is a flow chart illustrating exemplary steps that may be utilized for suppressing grating lobes using variable frequency as a function of beam steering, in accordance with various embodiments.

### DETAILED DESCRIPTION

Certain embodiments may be found in a method and system for suppressing grating lobes using variable frequency as a function of beam steering. Aspects of the present disclosure have the technical effect of improving ultrasound image quality due to reduction and/or avoidance of grating lobes by selecting a higher frequency for beams transmitted at smaller steering angles (i.e., near a center of the ultrasound image) and selecting a lower frequency for beams transmitted at larger steering angles (i.e., towards the edges and away from the center of the ultrasound image). Various embodiments have the technical effect of controlling the imaging frequency as a function of the beam steering angle. Certain embodiments have the technical effect of improving ultrasound image quality irrespective of the ultrasound scanner type (e.g., large, small, distributed between the ultrasound probe and the ultrasound machine with wired or wireless connection, wireless ultrasound systems, hardware based ultrasound systems, software based ultrasound systems, and the like) by selectively controlling the transmit frequency and/or the receive frequency as a function of the beam steering angle.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized, and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising”, “including”, or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode, which can be one-dimensional (1D), two-dimensional (2D), three-dimensional (3D), or four-dimensional (4D), and comprising B-mode, M-mode, CM-mode, CF-mode, PW Doppler, CW Doppler, Contrast Enhanced Ultrasound (CEUS), and/or sub-modes of B-mode and/or CF-mode such as Harmonic Imaging, Shear Wave Elasticity Imaging (SWEI), Strain Elastography, TVI, PDI, B-flow, MVI, UGAP, and the like.

Furthermore, the term processor or processing unit, as used herein, refers to any type of processing unit that can carry out the required calculations needed for the various embodiments, such as single or multi-core CPU, Accelerated Processing Unit (APU), Graphic Processing Unit (GPU), DSP, FPGA, ASIC or a combination thereof.

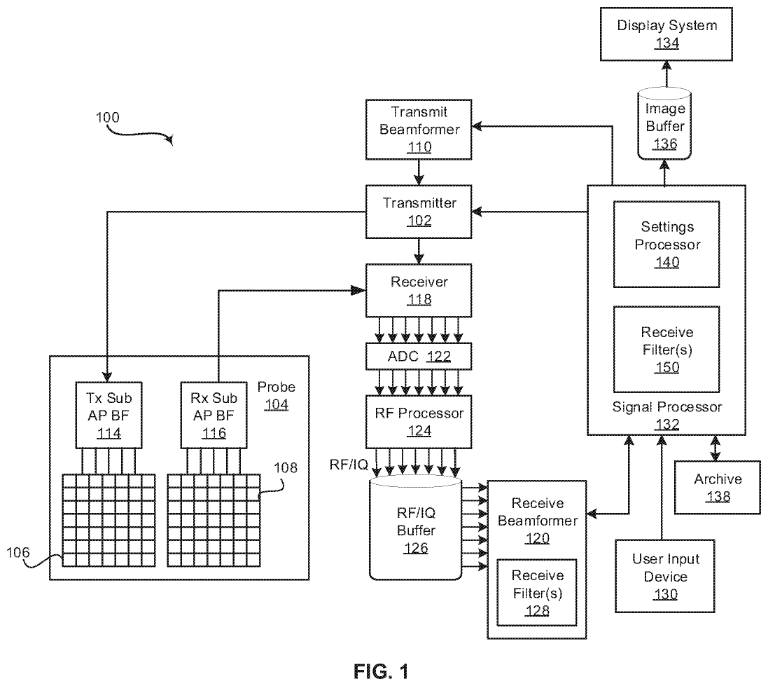

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to suppress grating lobes using variable frequency as a function of beam steering, in accordance with various embodiments. Referring to FIG. 1, there is shown an ultrasound system 100. The ultrasound system 100 comprises a transmitter 102, an ultrasound probe 104, a transmit beamformer 110, a receiver 118, a receive beamformer 120, A/D converters 122, a RF processor 124, a RF/IQ buffer 126, a user input device 130, a signal processor 132, an image buffer 136, a display system 134, and an archive 138. The ultrasound system 100 may be a standard console, a miniaturized ultrasound system, wired or wireless ultrasound system, and/or any ultrasound system capable of transmitting and receiving acoustic energy from a plurality of transducer elements and at a plurality of directions. In certain embodiments, the transmitter 102 and/or transmit beamformer 110 may embedded in the ultrasound probe 104.

The transmitter 102 may comprise suitable logic, circuitry, interfaces and/or code that may be operable to drive an ultrasound probe 104. The transmitter 102 may be configured to receive transmit settings for driving the ultrasound probe 104 from the signal processor 132 as described below. For example, the transmitter 102 may receive transmit settings such as a transmit frequency, waveform shape, bandwidth, and/or any suitable transmit settings from the signal processor 132. In various embodiments, the transmitter 102 may be configured to vary the transmit frequency as a function of a beam steering angle of a transmitted beam (e.g., transmit frequency decreases as a beam steering angle increases). In an exemplary embodiment, the transmit frequency may be fixed if the ultrasound system 100 is varying only the receive frequency as a function of the beam steering angle.

The ultrasound probe 104 may be a phased array, linear array, curved array, or any suitable shape or combination of shapes. The ultrasound probe 104 may comprise an array of transducer elements, such as piezoelectric elements, micromachined elements, piezoelectric micromachined ultrasound transducers (PMUT) elements, capacitive micromachined ultrasound transducers (CMUT) elements, and/or any suitable transducer elements capable of converting control signals to acoustic energy and converting acoustic energy to ultrasound signals. The ultrasound probe 104 may comprise a group of transmit transducer elements 106 and a group of receive transducer elements 108, that normally constitute the same elements. The group of transmit transducer elements 106 may emit ultrasonic signals into a target. In a representative embodiment, the ultrasound probe 104 may be operable to acquire ultrasound image data covering at least a substantial portion of an anatomy, such as a heart, fetus, blood vessels, pelvic region, or any suitable anatomical region.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals (i.e., transmit beams) into a region of interest (e.g., human, animal, underground cavity, physical structure and the like). In various embodiments, the transmit sub-aperture beamformer 114 may not be included. The transmit beamformer 110 may be configured to receive a transmit grid from the signal processor 132 as described below. The transmit grid defines the beam center trajectories of the transmit beams and is used to determine the transmit delay for each transducer element to control a beam steering angle of a transmitted beam. The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, which undergo sub-aperture beamforming by a receive sub-aperture beamformer 116 and are then communicated to a receiver 118. In various embodiments, the receive sub-aperture beamformer 116 may not be included. The receiver 118 may comprise suitable logic, circuitry, interfaces and/or code that may be operable to receive the signals from the receive sub-aperture beamformer 116 and/or receive transducer elements 108. The analog signals may be communicated to one or more of the plurality of A/D converters 122.

The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118 or in the probe 104.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

The receive beamformer 120 may comprise suitable logic, circuitry, interfaces and/or code that may be operable to perform digital beamforming processing to, for example, sum the delayed channel signals received from the RF processor 124 via the RF/IQ buffer 126 and output a beam summed signal. The resulting processed information may be the beam summed signal that is output from the receive beamformer 120 and communicated to the signal processor 132. The receive beamformer 120 may be configured to receive a receive frequency from the signal processor 132 that varies as a function of the beam steering angle (e.g., receive frequency decreases as a beam steering angle increases). The receive beamformer 120 may comprise receive filter(s) 128 configured to filter the ultrasound signals prior to beamforming based on the receive frequency. In accordance with some embodiments, the receiver 118, the plurality of A/D converters 122, the RF processor 124, the receive filter(s) 128, and the beamformer 120 may be integrated into a single beamformer, which may be digital. In various embodiments, the ultrasound system 100 comprises a plurality of receive beamformers 120.

The user input device 130 may be utilized to input patient data, scan parameters, settings, select protocols and/or templates, and the like. In an exemplary embodiment, the user input device 130 may be operable to configure, manage and/or control operation of one or more components and/or modules in the ultrasound system 100. In this regard, the user input device 130 may be operable to configure, manage and/or control operation of the transmitter 102, the ultrasound probe 104, the transmit beamformer 110, the receiver 118, the receive beamformer 120, the RF processor 124, the RF/IQ buffer 126, the signal processor 132, the image buffer 136, the display system 134, and/or the archive 138. The user input device 130 may include button(s), rotary encoder(s), a touchscreen, motion tracking, voice recognition, a mousing device, keyboard, camera and/or any other device capable of receiving a user directive. In certain embodiments, one or more of the user input devices 130 may be integrated into other components, such as the display system 134 or the ultrasound probe 104, for example. As an example, the user input device 130 may include a touchscreen display.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), or any suitable device for storing images and related information.

The signal processor 132 may be one or more processing units, microprocessors, microcontrollers, GPU, and/or the like. The signal processor 132 may be an integrated component, or may be distributed across various locations, for example. In an exemplary embodiment, the signal processor 132 may comprise a settings processor 140 and receive filter(s) 150. The signal processor 132 may be capable of receiving input information from a user input device 130 and/or archive 138, generating an output displayable by a display system 134, and manipulating the output in response to input information from a user input device 130, among other things. The signal processor 132, settings processor 140, and receive filter(s) 150 may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates range from 20-120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data but it can also store less. The frames of ultrasound scan data are stored in a manner to facilitate retrieval thereof according to its order or time of acquisition. The image buffer 136 may be embodied as any known data storage medium.

The signal processor 132 may include a settings processor 140 that comprises suitable logic, circuitry, interfaces and/or code that may be operable to setup a transmit grid, select a transmit waveform and transmit frequency, and select a receive frequency for an ultrasound scan. For example, the settings processor 140 may communicate with the transmit beamformer 110 to provide a transmit grid defining the beam trajectories of the transmit beams such that the transmit beamformer 110 can determine the transmit delay for each transducer element to control a beam steering angle of a transmitted beam. As another example, the settings processor 140 may communicate with the transmitter 102 to provide transmit settings, such as a transmit frequency, waveform shape, bandwidth, and the like for application by the transmitter 102, such that the transmitter 102 can vary the transmit frequency as a function of a beam steering angle of a transmitted beam. Furthermore, the settings processor 140 may communicate with receive filter(s) 128 of the receive beamformer 120 and/or control receive filter(s) 150 of the signal processor 132 to provide a receive frequency that varies as a function of the beam steering angle of the transmit and/or receive beam. The selection of various aspects of the transmit grid, imaging frequency (i.e., transmit and/or receive frequencies), waveform shape, bandwidth, and the like may be based in part on an imaging mode and imaging settings within the imaging mode.

The settings processor 140 controls the transmit and receive frequency to maximize frequency and image resolution in a center region of an ultrasound image while reducing and/or avoiding grating lobes by reducing imaging frequency for larger steering angles. The control of the imaging frequencies as a function of the beam steering angle may be provided by the settings processor 140 at set-up (i.e., prior to an ultrasound scan) and/or dynamically during the ultrasound scan. In various embodiments, the settings processor 140 may be configured to control variation of the transmit frequency only, the receive frequency only, or both the transmit and receive frequencies. For example, the settings processor 140 may control the transmit beamformer 110 and transmitter 102 to provide transmit frequencies that decrease as a beam steering angle increases, while maintaining a fixed receive frequency provided by the receive filter(s) 128, 150 of the receive beamformer 120 and/or signal processor 132. As another example, the settings processor 140 may control the transmit beamformer 110 and transmitter 102 to provide a fixed transmit frequency while controlling the receive filter(s) 128, 150 of the receive beamformer 120 and/or signal processor 132 to provide receive frequencies that decrease as a beam steering angle increases. As another example, the settings processor 140 may control the transmit beamformer 110 and transmitter 102 to provide transmit frequencies that decrease as a beam steering angle increases, while controlling the receive filter(s) 128, 150 of the receive beamformer 120 and/or signal processor 132 to provide receive frequencies that decrease as a beam steering angle increases. The functional relationship between the imaging frequencies and beam steering angles may be linear, non-linear, monotonic, non-monotonic, and/or stepwise controlled. The transmit and/or receive frequency lowering as controlled by the settings processor 140 depends on image width, imaging frequency at a center region of the ultrasound image, and transducer element size. The transmit and/or receive frequency lowering provides a tradeoff between image resolution and grating lobe suppression. Specifically, the transmit and/or receive frequency lowering provides a highest image quality in a center part of the ultrasound image while avoiding or suppressing a level of grating lobes at larger steering angles by gradually lowering the transmit and/or receive frequency as a function of beam steering angle. The reduction of image resolution for largely steered beams is an attractive tradeoff compared to the presence of grating lobe artifacts in the ultrasound image.

The signal processor 132 may include receive filter(s) 150 that comprises suitable logic, circuitry, interfaces and/or code that may be operable to apply a receive frequency provided by the settings processor 140 to beamformed ultrasound signals received from the receive beamformer 120. For example, the receive filter(s) 150 may be configured to filter the beamformed ultrasound signals based on a receive frequency that varies as a function of the beam steering angle (i.e., receive frequency decreases as a beam steering angle increases). In various embodiments, the receive filtering may be applied by the receive filter(s) 128 of the receive beamformer 120, the receive filter(s) 150 of the signal processor 132, or both of the receive filter(s) of the receive beamformer 120 and the receive filter(s) 150 of the signal processor 132. In certain embodiments, the receive filtering may be done at any filtering steps in the processing chain, or by varying the demodulation frequency as a function of the beam steering angle to obtain a desired frequency band. In an exemplary embodiment, the receive frequency applied by the receive filter(s) 128, 150 may be fixed if the ultrasound system 100 is varying only the transmit frequency as a function of the beam steering angle.

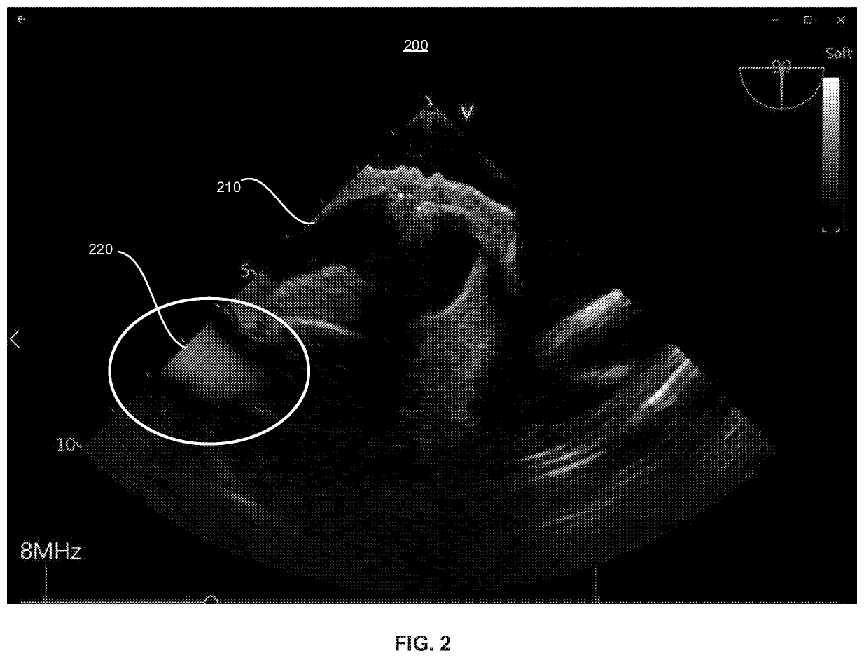

FIG. 2 is an exemplary display 200 of an ultrasound image 210 having a grating lobe 220, in accordance with various embodiments. Referring to FIG. 2, the display 200 comprises an ultrasound image 210 having a grating lobe 220. The grating lobe 220 appears as a shadow or cloud in the ultrasound image 210. The presence and level of the grating lobe 220 depends on probe element spacing and imaging frequency. To avoid the grating lobe 220, the imaging frequency should be reduced. In the example of FIG. 2, the imaging frequency is too high as compared to the beam steering angle in question, causing the grating lobe 220 to appear above a maximum beam steering angle.

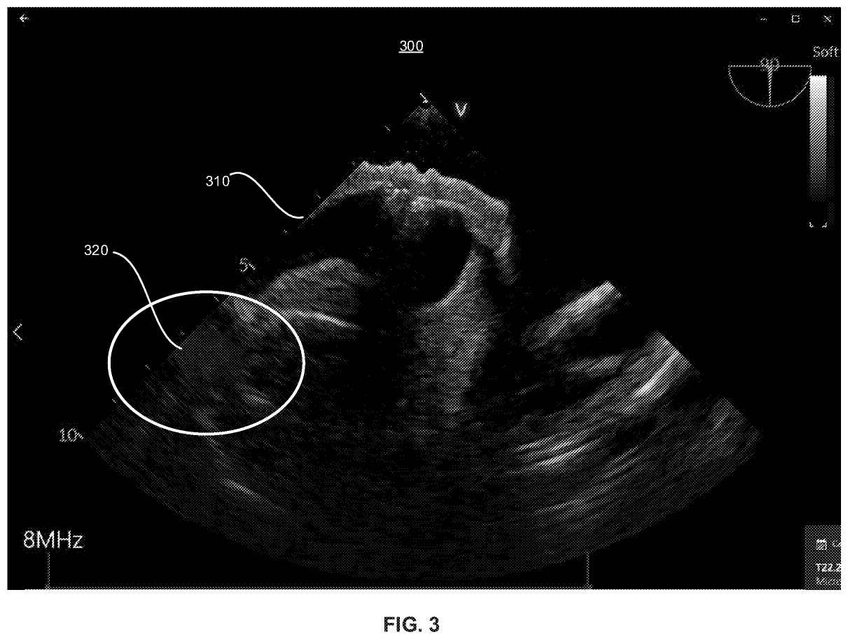

FIG. 3 is an exemplary display 300 of an ultrasound image 310 with a suppressed grating lobe as compared to FIG. 2, in accordance with various embodiments. Referring to FIG. 3, the display 300 comprises an ultrasound image 310. The region 320 of the ultrasound image 310 that included a grating lobe 220 in FIG. 2 no longer includes the grating lobe 220 because the imaging frequency has been reduced such that the element spacing is a sufficiently small fraction of the wavelength for the beam steering angles in question.

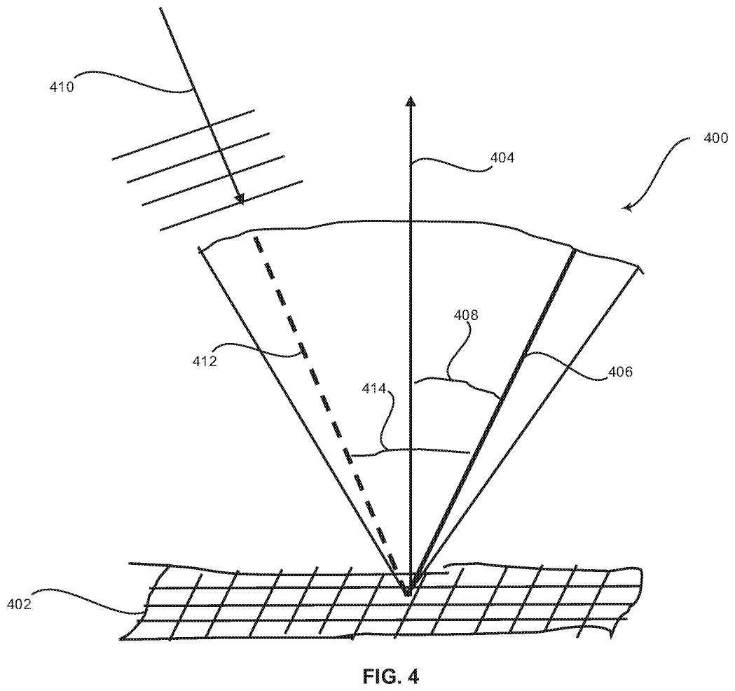

FIG. 4 is an illustration 400 of an exemplary ultrasound beam transmission 406 at a beam steering angle 408 and received incoming energy 410, 412 providing the grating lobe 220 of FIG. 2, in accordance with various embodiments. Referring to FIG. 4, an ultrasound beam 406 is transmitted from an ultrasound transducer 402 at a beam steering angle 408 with respect to a central axis 404. The terms large and small in relation to the beam steering angle as used herein refers to the absolute value of the beam steering angle. For example, a large beam steering angle indicates the beam steering angle is farther from the central axis and a small beam steering angle indicates the beam steering angle is closer to the central axis, irrespective of sign (i.e., positive or negative beam steering angles). Incoming energy 410, 412 is received at a grating lobe angle offset 414 with respect to the transmitted ultrasound beam 406 causing the appearance of a grating lobe 220 as shown in FIG. 2. The appearance of the grating lobe is due to the imaging frequency reaching above a limit defined by the element spacing being a fraction of the wavelength, and the beam steering angle 408 being above a maximum beam steering angle.

Referring again to FIG. 1, the display system 134 may be any device capable of communicating visual information to a user. For example, a display system 134 may include a liquid crystal display, a light emitting diode display, and/or any suitable display or displays. The display system 134 can be operable to present the ultrasound images 210, 310, and/or any suitable information.

The archive 138 may be one or more computer-readable memories integrated with the ultrasound system 100 and/or communicatively coupled (e.g., over a network) to the ultrasound system 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores instructions for setting a transmit and/or receive frequency as a function of beam steering angle 408, setting-up a transmit grid of an ultrasound scan, and/or selecting a transmit waveform of an ultrasound scan, for example.

Components of the ultrasound system 100 may be implemented in software, hardware, firmware, and/or the like. The various components of the ultrasound system 100 may be communicatively linked. Components of the ultrasound system 100 may be implemented separately and/or integrated in various forms. For example, the display system 134 and the user input device 130 may be integrated as a touchscreen display.

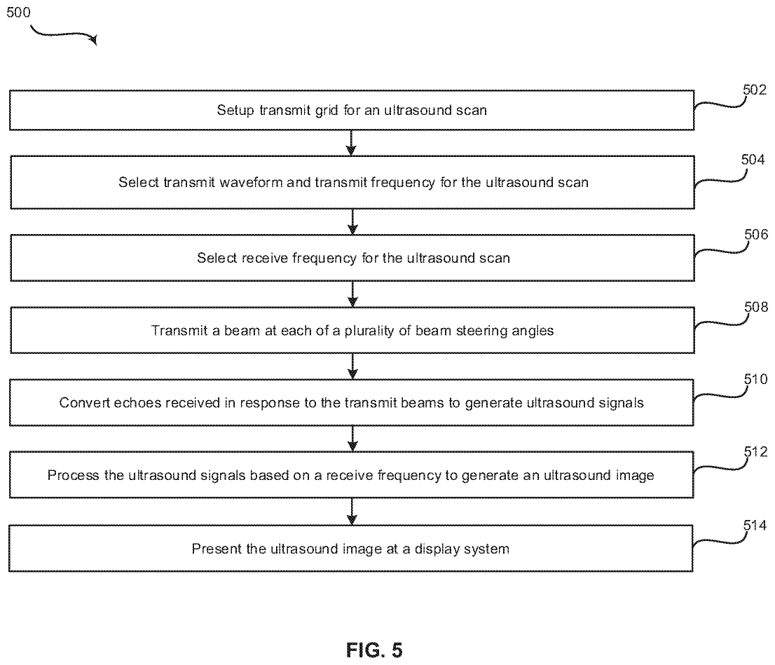

FIG. 5 is a flow chart 500 illustrating exemplary steps 502-514 that may be utilized for suppressing grating lobes 220 using variable frequency as a function of beam steering, in accordance with various embodiments. Referring to FIG. 5, there is shown a flow chart 500 comprising exemplary steps 502 through 514. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 502, a signal processor 132 of an ultrasound system 100 may setup a transmit grid for an ultrasound scan. For example, a settings processor 140 of the signal processor 132 may be configured to communicate with a transmit beamformer 110 of the ultrasound system 100 to provide a transmit grid defining the beam trajectories of the transmit beams such that the transmit beamformer 110 can determine the transmit delay for each transducer element to control a beam steering angle 408 of a transmitted beam 406.

At step 504, the signal processor 132 of the ultrasound system 100 may select a transmit waveform and transmit frequency for the ultrasound scan. For example, the settings processor 140 of the signal processor 132 may communicate with the transmitter 102 to provide transmit settings, such as a transmit frequency, waveform shape, bandwidth, and the like for application by the transmitter 102, such that the transmitter 102 can vary the transmit frequency as a function of a beam steering angle of a transmitted beam.

At step 506, the signal processor 132 of the ultrasound system 100 may select a receive frequency for the ultrasound scan. For example, the settings processor 140 of the signal processor 132 may communicate with receive filter(s) 128 of the receive beamformer 120 and/or control receive filter(s) 150 of the signal processor 132 to provide a receive frequency that varies as a function of the beam steering angle of the transmitted beam.

At step 508, an ultrasound probe 104 of the ultrasound system 100 may transmit a beam at each of a plurality of beam steering angles. For example, the ultrasound probe 104 may transmit beams, one at a time, in a plurality of predetermined beam steering angles. In various embodiments, the settings processor 140 may be configured to control the transmit frequency applied by the transmitter 102 such that a transmit frequency is highest when the beam steering angle is the smallest. The transmit frequency may be decreased as the beam steering angle increases. In an exemplary embodiment, the transmit frequency may be fixed when the settings processor 140 is configured to vary only the receive frequency. Additionally and/or alternatively, the settings processor 140 may be configured to vary both the transmit and receive frequencies.

At step 510, the ultrasound probe 104 of the ultrasound system 100 may convert echoes received in response to the transmit beams to generate ultrasound signals. For example, the ultrasound beams transmitted at step 508 may be back-scattered from structures in the object of interest and the echoes are received by the receive transducer elements 108, which may be operable to convert the received echoes into analog signals. The analog signals may be converted to digital signals, which are demodulated to form I/Q data pairs that are representative of the corresponding echo signals.

At step 512, the signal processor 132 and/or a receive beamformer 120 of the ultrasound system 100 may process the ultrasound signals based on a receive frequency to generate an ultrasound image. For example, the receive beamformer 120 may be configured to perform digital beamforming processing to, for example, sum the delayed channel signals and output a beam summed signal. The resulting processed information may be the beam summed signal that is output from the receive beamformer 120 and communicated to the signal processor 132. The signal processor 132 may be configured to process ultrasound scan data for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. The receive beamformer 120 and/or signal processor 132 may comprise receive filter(s) 128, 150 configured to filter the ultrasound signals prior to and/or after beamforming based on a receive frequency received from the settings processor 140 that varies as a function of the beam steering angle. In various embodiments, the settings processor 140 may be configured to control the receive frequency applied by the receive filter(s) 128, 150 such that a receive frequency is high when the beam steering angle is small. The receive frequency may be decreased as the beam steering angle increases. In an exemplary embodiment, the receive frequency may be fixed when the settings processor 140 is configured to vary only the transmit frequency. Additionally and/or alternatively, the settings processor 140 may be configured to vary both the transmit and receive frequencies.

At step 514, the signal processor 132 of the ultrasound system 100 may cause a display system 134 to present the ultrasound image 310. For example, the ultrasound image 310 generated by the ultrasound system 100 may be presented at the display system 134. The ultrasound image 310 may have a highest image resolution toward a center of the ultrasound image 310. Although image resolution may decrease toward the outer edges of the ultrasound image 310, grating lobes are reduced and/or avoided.

Aspects of the present disclosure provide a method 500 and system 100 for suppressing grating lobes 220 using variable frequency as a function of beam steering. In accordance with various embodiments, the method 500 may comprise transmitting 508, by an ultrasound probe 104 of an ultrasound system 100, one or more beams 406 each at a beam steering angle 408. The method 500 may comprise converting 510, by the ultrasound probe 104, received echoes to generate ultrasound signals corresponding to the one or more beams 406. The method 500 may comprise processing 512, by at least one processor 132, 150 and/or a receive beamformer 120, 128 of the ultrasound system 100, the ultrasound signals to generate an ultrasound image 310. The method 500 may comprise causing 514, by the at least one processor 132, a display system 134 of the ultrasound system 100 to present the ultrasound image 310. A transmit frequency and/or a receive frequency is selected based on the beam steering angle 408 of each of the one or more beams 406. A larger beam steering angle 408 corresponds with a lower transmit frequency and/or a lower receive frequency. A smaller beam steering angle 408 corresponds with a higher transmit frequency and/or a higher receive frequency.

In an exemplary embodiment, the receive frequency is fixed and the transmit frequency is selected based on the beam steering angle 408 of each of the one or more beams 406. In a representative embodiment, the transmit frequency is fixed and the receive frequency is selected based on the beam steering angle 408 of each of the one or more beams 406. In various embodiments, both the transmit frequency and the receive frequency are selected based on the beam steering angle 408 of each of the one or more beams 406. In certain embodiments, the receive frequency is applied by at least one receive filter 128 of the receive beamformer 120, 128 based on the beam steering angle 408 of each of the one or more beams 406. In an exemplary embodiment, the receive frequency is applied by at least one receive filter 150 of the at least one processor 132, 150 based on the beam steering angle 408 of each of the one or more beams 406. In a representative embodiment, the method 500 comprises providing 502, by the at least one processor 132, 140, a transmit grid defining the beam steering angle 408 of each of the one or more beams 406 to a transmit beamformer 110 of the ultrasound system 100. In various embodiments, the method 500 comprises providing 504, by the at least one processor 132, 140, the transmit frequency corresponding to the beam steering angle 408 of each of the one or more beams 406 to a transmitter 102 of the ultrasound system 100. In certain embodiments, the method 500 comprises providing 506, by the at least one processor 132, 140, the receive frequency corresponding to the beam steering angle 408 of each of the one or more beams 406 to the receive beamformer 120, 128. In an exemplary embodiment, the method 500 comprises providing 504, by the at least one processor 132, 140, transmit waveform settings to a transmitter 102 of the ultrasound system 100.

Various embodiments provide a system 100 for suppressing grating lobes 220 using variable frequency as a function of beam steering. The ultrasound system 100 may comprise an ultrasound probe 104, at least one processor 132, 140, 150, a receive beamformer 120, 128, and a display system 134. The ultrasound probe 104 may be configured to transmit one or more beams 406 each at a beam steering angle 408. The ultrasound probe 104 may be configured to convert received echoes to generate ultrasound signals corresponding to the one or more beams 406. The at least one processor 132, 150 and/or the receive beamformer 120, 128 may be configured to process the ultrasound signals to generate an ultrasound image 310. The display system 134 may be configured to present the ultrasound image 310. A transmit frequency and/or a receive frequency is selected based on the beam steering angle 408 of each of the one or more beams 406. A larger beam steering angle 408 corresponds with a lower transmit frequency and/or a lower receive frequency. A smaller beam steering angle 408 corresponds with a higher transmit frequency and/or a higher receive frequency.

In a representative embodiment, the receive frequency is fixed and the transmit frequency is selected based on the beam steering angle 408 of each of the one or more beams 406. In various embodiments, the transmit frequency is fixed and the receive frequency is selected based on the beam steering angle 408 of each of the one or more beams 406. In certain embodiments, both the transmit frequency and the receive frequency are selected based on the beam steering angle 408 of each of the one or more beams 406. In an exemplary embodiment, the receive beamformer 120, 128 comprises at least one receive filter 128. The at least one receive filter 128 is configured to apply the receive frequency based on the beam steering angle 408 of each of the one or more beams 406. In a representative embodiment, the at least one processor 132, 140, 150 comprises at least one receive filter 150. The at least one receive filter 150 is configured to apply the receive frequency based on the beam steering angle 408 of each of the one or more beams 406. In various embodiments, the ultrasound system 100 comprises a transmit beamformer 110. The at least one processor 132, 140 is configured to provide a transmit grid defining the beam steering angle 408 of each of the one or more beams 406 to the transmit beamformer 110. In certain embodiments, the ultrasound system 100 comprises a transmitter 102. The at least one processor 132, 140 is configured to provide the transmit frequency corresponding to the beam steering angle 408 of each of the one or more beams 406 to the transmitter 102. In an exemplary embodiment, the at least one processor 132, 140 is configured to provide the receive frequency corresponding to the beam steering angle 408 of each of the one or more beams 406 to the receive beamformer 120, 128. In a representative embodiment, the ultrasound system 100 comprises a transmitter 102. The at least one processor 132, 140 is configured to provide transmit waveform settings to the transmitter 102.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” and/or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for suppressing grating lobes using variable frequency as a function of beam steering.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A method comprising:
transmitting, by an ultrasound probe of an ultrasound system, one or more beams each at a beam steering angle;
converting, by the ultrasound probe, received echoes to generate ultrasound signals corresponding to the one or more beams;
processing, by at least one processor and/or a receive beamformer of the ultrasound system, the ultrasound signals to generate an ultrasound image; and
causing, by the at least one processor, a display system of the ultrasound system to present the ultrasound image,
wherein:
a transmit frequency and/or a receive frequency is selected based on the beam steering angle of each of the one or more beams,
a larger beam steering angle corresponds with a lower transmit frequency and/or a lower receive frequency, and
a smaller beam steering angle corresponds with a higher transmit frequency and/or a higher receive frequency.

2. The method of claim 1, wherein the receive frequency is fixed and the transmit frequency is selected based on the beam steering angle of each of the one or more beams.

3. The method of claim 1, wherein the transmit frequency is fixed and the receive frequency is selected based on the beam steering angle of each of the one or more beams.

4. The method of claim 1, wherein both the transmit frequency and the receive frequency are selected based on the beam steering angle of each of the one or more beams.

5. The method of claim 1, wherein the receive frequency is applied by at least one receive filter of the receive beamformer based on the beam steering angle of each of the one or more beams.

6. The method of claim 1, wherein the receive frequency is applied by at least one receive filter of the at least one processor based on the beam steering angle of each of the one or more beams.

7. The method of claim 1, comprising providing, by the at least one processor, a transmit grid defining the beam steering angle of each of the one or more beams to a transmit beamformer of the ultrasound system.

8. The method of claim 1, comprising providing, by the at least one processor, the transmit frequency corresponding to the beam steering angle of each of the one or more beams to a transmitter of the ultrasound system.

9. The method of claim 1, comprising providing, by the at least one processor, the receive frequency corresponding to the beam steering angle of each of the one or more beams to the receive beamformer.

10. The method of claim 1, comprising providing, by the at least one processor, transmit waveform settings to a transmitter of the ultrasound system.

11. An ultrasound system comprising:
an ultrasound probe configured to:
transmit one or more beams each at a beam steering angle; and
convert received echoes to generate ultrasound signals corresponding to the one or more beams;
at least one processor and a receive beamformer, wherein the at least one processor and/or the receive beamformer is configured to process the ultrasound signals to generate an ultrasound image; and
a display system configured to present the ultrasound image,
wherein:
a transmit frequency and/or a receive frequency is selected based on the beam steering angle of each of the one or more beams,
a larger beam steering angle corresponds with a lower transmit frequency and/or a lower receive frequency, and
a smaller beam steering angle corresponds with a higher transmit frequency and/or a higher receive frequency.

12. The ultrasound system of claim 11, wherein the receive frequency is fixed and the transmit frequency is selected based on the beam steering angle of each of the one or more beams.

13. The ultrasound system of claim 11, wherein the transmit frequency is fixed and the receive frequency is selected based on the beam steering angle of each of the one or more beams.

14. The ultrasound system of claim 11, wherein both the transmit frequency and the receive frequency are selected based on the beam steering angle of each of the one or more beams.

15. The ultrasound system of claim 11, wherein:
the receive beamformer comprises at least one receive filter, and
the at least one receive filter is configured to apply the receive frequency based on the beam steering angle of each of the one or more beams.

16. The ultrasound system of claim 11, wherein:
the at least one processor comprises at least one receive filter, and
the at least one receive filter is configured to apply the receive frequency based on the beam steering angle of each of the one or more beams.

17. The ultrasound system of claim 11, comprising a transmit beamformer, wherein the at least one processor is configured to provide a transmit grid defining the beam steering angle of each of the one or more beams to the transmit beamformer.

18. The ultrasound system of claim 11, comprising a transmitter, wherein the at least one processor is configured to provide the transmit frequency corresponding to the beam steering angle of each of the one or more beams to the transmitter.

19. The ultrasound system of claim 11, wherein the at least one processor is configured to provide the receive frequency corresponding to the beam steering angle of each of the one or more beams to the receive beamformer.

20. The ultrasound system of claim 11, comprising a transmitter, wherein the at least one processor is configured to provide transmit waveform settings to the transmitter.
